(19) United States
(12) Patent Application Publication
(10) Pub. No.: US 2017/0035355 A1
(43) Pub. Date: Feb. 9, 2017

(54) INTELLIGENT WEARABLE DEVICE AND METHOD FOR PHYSICAL SIGN MEASUREMENT

(71) Applicant: Boe Technology Group Co., Ltd., Beijing (CN)

(72) Inventor: Ruisi Chen, Beijing (CN)

(73) Assignee: Boe Technology Group Co., Ltd., Beijing (CN)

(21) Appl. No.: 14/913,267

(22) PCT Filed: Aug. 10, 2015
(86) PCT No.: PCT/CN2015/086449

§ 371 Date: Feb. 19, 2016

(30) Foreign Application Priority Data
Mar. 26, 2015 (CN) 201510137547.0

(51) Int. Cl. A61B 5/00; A61B 5/021; A61B 5/024

(52) U.S. Cl. CPC A61B 5/6843; A61B 5/6801; A61B 5/02438; A61B 5/02416; A61B 5/742; A61B 5/681; A61B 5/02108

(57) ABSTRACT

An intelligent wearable device and a method for physical sign measurement, comprising a photoelectric sensor, an inflatable ring, an air valve and a control unit. When the valve is in an open state, the inflatable ring can be inflated and deflated alternately. When the air valve is in a closed state, inflation or deflation of the inflatable ring is stopped. The photoelectric sensor is located at an inner side of the inflatable ring for measuring physical signs, with the control unit is connected to both the photoelectric sensor and the air valve for controlling the state of open and close of the air valve according to the physical sign parameters measured by the photoelectric sensor. The device measures physical signs on the basis of automatically controlling the air valve, where the photoelectric sensor contacts the vascular wall, and as a result, improves accuracy the measurements.

### RELATED APPLICATIONS

The present application is the U.S. national phase entry of PCT/CN2015/086449 with an International filing date of Aug. 10, 2015, which claims the benefit of Chinese Application No. 201510137547.0, filed Mar. 26, 2015, the entire disclosures of which are incorporated herein by reference.

### TECHNICAL FIELD

The present invention relates to the field of intelligent wearing, in particular to an intelligent wearable device and a method for physical sign measurement.

### BACKGROUND OF THE INVENTION

Photo PlethysmoGraphy (PPG) is a non-invasive method for measuring physical sign parameters. It uses a photoelectric sensor to trace in real time the quantity of light absorbed by a measured part of a human body so as to obtain blood volume changes of the micrangium in a cardiac cycle, thereby obtaining a pulse wave from which parameters like heart rate and blood oxygen can be obtained.

In the PPG of the prior art, light emitted by the photoelectric sensor is absorbed by the skin, blood, etc. and is reflected back to the photoelectric sensor, and upon receiving the light signal, the photoelectric sensor measures a light intensity and calculates a light absorption quantity according to the light intensity. The blood volume in the skin changes in a pulsating pattern under the effect of the heart, the blood has the largest light absorption quantity when the heart is systaltic, so the measured light intensity is the lowest; the blood has the smallest light absorption quantity when the heart is diastolic, so the measured light intensity is the highest; thus the light signal received by the photoelectric sensor changes accordingly in a pulsating pattern, so that a pulse wave can be obtained therefrom.

In the above-mentioned process, however, a pressure at the position where the photoelectric sensor contacts the vascular wall will influence the light signal detected by the photoelectric sensor. When the pressure at the position of contact is too large or too small, the light signal detected by the photoelectric sensor will be weakened, thus influencing the result of measurement and causing inaccurate measurement result.

### SUMMARY OF THE INVENTION

In order to overcome the above-mentioned problem and defect in the prior art, the present invention provides an intelligent wearable device and a method for physical sign measurement so as to improve accuracy of physical sign measurement.

According to a first aspect of the present invention, an intelligent wearable device is provided, which comprises: a photoelectric sensor, an inflatable ring, an air valve and a control unit, wherein the air valve is within the inflatable ring, and when the air valve is in an open state, the inflatable ring can be inflated and deflated alternately, and when the air valve is in a closed state, inflation or deflation of the inflatable ring is stopped; the photoelectric sensor is located at an inner side of the inflatable ring for measuring physical signs; and the control unit is connected to the photoelectric sensor and the air valve for controlling the state of open and close of the air valve according to the physical sign parameters measured by the photoelectric sensor.

The beneficial effects of the intelligent wearable device according to the present invention lie in that it measures physical signs on the basis of automatically controlling the air valve during operation, wherein an amount of air inflated into the inflatable ring can be regulated by controlling the state of open and close of the air valve, thereby properly controlling the pressure formed at the position where the photoelectric sensor contacts the vascular wall, so as to reduce influence to the result of physical sign measurement caused by the pressure formed at the position where the photoelectric sensor contacts the vascular wall, and as a result, accuracy of the result of measurement can be improved.

In one embodiment, in the case where the air valve is in an open state, the control unit can also be used for closing the air valve when the pulse wave signal measured by the photoelectric sensor is the strongest. Wherein, the pulse wave signal being the strongest may specifically mean that the amplitude of the pulse wave is the largest.

In some of those embodiments, the control unit can be further used for, during inflation of the inflatable ring, determining an optimal inflating volume for the inflatable ring when the pulse wave signal is the strongest, and for, during deflation of the inflatable ring, closing the air valve when the inflatable ring has reached the optimal inflating volume.

Similarly, the control unit can be further used for, during deflation of the inflatable ring, determining an inflating volume for the inflatable ring when the pulse wave signal is the strongest, and for, during inflation of the inflatable ring, closing the air valve when the inflatable ring has reached the inflating volume.

In one, the intelligent wearable device may further comprises a dial plate, which is connected to the photoelectric sensor and the inflatable ring for displaying various physical sign parameters and data measured by the photoelectric sensor, and the control unit is in the dial plate.

According to a second aspect of the present invention, a method for physical sign measurement is provided, which uses the intelligent wearable device according to the present invention, the method comprises: controlling the air valve to be in an open state by the control unit; measuring the physical signs in real time by the photoelectric sensor; and determining, by the control unit, whether the air valve should be closed according to the physical sign parameters measured by the photoelectric sensor.

Preferably, the step of determining, by the control unit, whether the air valve should be closed according to the physical sign parameters measured by the photoelectric sensor may include: closing the air valve by the control unit when the pulse wave signal measured by the photoelectric sensor is the strongest. Wherein, the pulse wave signal being the strongest may specifically mean that the amplitude of the pulse wave is the largest.

More preferably, the step of closing the air valve by the control unit when the pulse wave signal measured by the photoelectric sensor is the strongest includes:
during inflation of the inflatable ring, determining, by the control unit, an optimal inflating volume for the inflatable ring when the pulse wave signal is the strongest, and during deflation of the inflatable ring, closing, by the control unit, the air valve when the inflatable ring has reached the optimal inflating volume; or
during deflation of the inflatable ring, determining, by the control unit, an optimal inflating volume for the inflatable ring when the pulse wave signal is the strongest, and during inflation of the inflatable ring, closing, by the control unit, the air valve when the inflatable ring has reached the optimal inflating volume.

By measuring physical signs on the basis of automatically controlling the air valve, it is possible to reduce the influence to the result of physical sign measurement caused by the pressure at the position where the photoelectric sensor contacts the vascular wall, and thus accuracy of the result of measurement can be improved.

### BRIEF DESCRIPTION OF THE DRAWINGS

In order to describe the technical solutions in the embodiments of the present invention more clearly, figures that need to be used in description of the embodiments will be introduced briefly below, obviously, the figures on the following descriptions are merely some embodiments of the present invention, and to those skilled in the art, other figures can be obtained from these figures without using any inventive skill.

FIG. 1 is a structural diagram of an intelligent wearable device provided by one embodiment of the present invention;

FIG. 2 is a structural diagram of an intelligent wearable device provided by another embodiment of the present invention;

FIG. 3 is a flow chart of a method for physical sign measurement provided by one embodiment of the present invention, which uses the intelligent wearable device according to the present invention; and

FIG. 4 is a flow chart of a method for physical sign measurement provided by another embodiment of the present invention, which uses the intelligent wearable device according to the present invention.

### DETAILED DESCRIPTION OF THE EMBODIMENTS

In order to enable a clearer understanding of the object, technical solutions and advantages of the present invention, embodiments of the present invention will be further described in detail below with reference to the figures.

FIG. 1 is a structural diagram of an intelligent wearable device provided by one embodiment of the present invention. As shown in FIG. 1, an intelligent wearable device according to one embodiment of the present invention comprises: a photoelectric sensor 11, an inflatable ring 12, an air valve 13 and a control unit 14;
wherein the air valve 13 is within the inflatable ring 12, and when the air valve 13 is in an open state, the inflatable ring 12 can be inflated and deflated alternately, and when the air valve 13 is in a closed state, inflation or deflation of the inflatable ring 12 is stopped. The photoelectric sensor 11 is located at an inner side of the inflatable ring 12 for measuring physical signs; and the control unit 14 is connected to the photoelectric sensor 11 and the air valve 13 respectively for controlling the state of open and close of the air valve 13 according to the physical sign parameters measured by the photoelectric sensor 11.

In the intelligent wearable device according to an embodiment of the present invention, physical signs are measured on the basis of automatically controlling the air valve 13, wherein an amount of air inflated into the inflatable ring 12 can be regulated by controlling the state of open and close of the air valve 13, thereby adjusting tightness between the photoelectric sensor 11 and the vascular wall and properly controlling the pressure formed at the position where the photoelectric sensor contacts the vascular wall, consequently, influence to the result of physical sign measurement caused by the pressure at the position where the photoelectric sensor 11 contacts the vascular wall can be reduced and accuracy of the result of measurement can be improved.

The intelligent wearable device provided in this embodiment can be worn on a human body for measuring physical signs. The intelligent wearable device can be designed as, for example, a smart watch or a smart wristband that can be worn on the wrist. In the intelligent wearable device according to the present embodiment, the physical signs that can be measured include various physiological parameters like heart rate, blood pressure, etc. The photoelectric sensor measures pulse waves to obtain corresponding physical sign parameters. The pulse wave is resulted from different quantities of the incident light and the reflected light because blood absorbs light spectrum. The pulse wave is a wave-form signal reflecting the pulse, and its frequency represents the pulse, its amplitude represents the strength of pulse.

In addition, the specific position of the air valve 13 at the inner side of the inflatable ring 12 may not be limited, as long as it is at the inner side of the ring. As shown in FIG. 1, the air valve 13 is located at the left side in the inflatable ring 12, but this is exemplary, and of course it can be located at the right side or lower side and so forth in the inflatable ring 12.

The inner side of the inflatable ring 12 refers to the side contacting the skin, which has a smaller radius, while the outer side refers to the side away from the skin, which has a larger radius. As shown in FIG. 1, the photoelectric sensor 11 is at the inner side of the inflatable ring 12 so as to be in direct contact with the skin to facilitate physical sign measurement. Preferably, in the intelligent wearable device according to the embodiment of the present invention, in the case where the air valve 13 is in an open state, the control unit 14 can also be used for closing the air valve 13 when the pulse wave signal measured by the photoelectric sensor 11 is the strongest.

In the process of operation of the intelligent wearable device as shown in FIG. 1, the air valve 13 is initially in an open state, and the inflatable ring 12 is inflated and deflated alternately. The inflation stops when the inflating volume reaches a preset maximum value, and the deflation stops when the inflating volume reaches a preset minimum value. The maximum and minimum values of inflating volume can be set as desired, and the specific values are not limited herein. When the control unit 14 closes the air valve 13, the air valve 13 stops inflation or deflation, and the inflating volume remains at the inflating volume when the air valve 13 is closed. When the pulse wave signal is the strongest, it is subjected to the least influence from the pressure at the position where the photoelectric sensor 11 contacts the vascular wall, and the inflating volume of the inflatable ring 12 is the optimal inflating volume at this time, and the control unit 14 then closes the air valve 13 to make the optimal inflating volume remain in the inflatable ring 12, thereby improving accuracy of the result of measurement by the photoelectric sensor 11.

It shall be noted that when a user wears the intelligent wearable device, the operation of determining the optimal inflating volume and closing the air valve may be performed only once, and the air valve does not need to be further adjusted any more. If the user takes off the intelligent wearable device and then re-wears it, the air valve needs to be re-adjusted to determine the optimal inflating volume, so as to guarantee accuracy of each time of measurement. Preferably, the air valve can be set to a default open state each time the intelligent wearable device is turned on, and the air valve is closed when the optimal inflating volume is reached, thus facilitating operation by the user.

Preferably, in the intelligent wearable device according to the present embodiment, the control unit 14 can also be used for, during inflation of the inflatable ring 12, determining an inflating volume (an optimal inflating volume) for the inflatable ring 12 when the pulse wave signal is the strongest, and for, during deflation of the inflatable ring 12, closing the air valve 13 when the inflatable ring 12 has reached the optimal inflating volume. Specifically, the optimal inflating volume can be determined in the following manner: recording pulse wave signal strengths and inflating volumes of the inflatable ring 12 corresponding to various moments (i.e. multiple moments that fully cover the time interval for the inflation process) during the inflation, then comparing the recorded pulse wave signal strengths to obtain the moment in which the pulse wave signal is the strongest and recording the inflating volume corresponding to the moment as the optimal inflating volume. In addition, during inflation, signal strengths and inflating volumes at multiple moments may be recorded, and then a curve fitting method is used to obtain a curve of approximate function relationship between the pulse wave signal strengths and the inflating volumes of the inflatable ring 12 (the degree of approximation of the curve of function relationship can reach a predetermined precision as long as there are enough moments and the intervals between the adjacent moments are small enough), thus the optimal inflating volume corresponding to the moment in which the pulse wave signal is the strongest can be calculated. Since inflation and deflation are carried out alternately, and the optimal inflating volume is determined when the inflation stops, in the following deflation process, the air valve can be controlled according to the optimal inflating volume, and the air valve is closed when deflating to the optimal inflating volume.

Similarly, in the intelligent wearable device according to the present embodiment, the control unit 14 can also be used for, during deflation of the inflatable ring 12, determining an optimal inflating volume for the inflatable ring 12 when the pulse wave signal is the strongest, and for, during inflation of the inflatable ring 12, closing the air valve 13 when the inflatable ring 12 has reached the optimal inflating volume. Specifically, the optimal inflating volume can be determined in the following manner: recording pulse wave signal strengths and inflating volumes of the inflatable ring 12 corresponding to various moments during the deflation, then comparing the recorded pulse wave signal strengths to obtain the moment in which the pulse wave signal is the strongest and recording the inflating volume corresponding to the moment as the optimal inflating volume. Since deflation and inflation are carried out alternately, and the optimal inflating volume is determined when the deflation stops, in the following inflation process, the air valve can be controlled according to the optimal inflating volume, and the air valve is closed when inflating to the optimal inflating volume.

In the intelligent wearable device according to the present embodiment, preferably, the pulse wave signal being the strongest as mentioned above may specifically mean that the amplitude of the pulse wave is the largest.

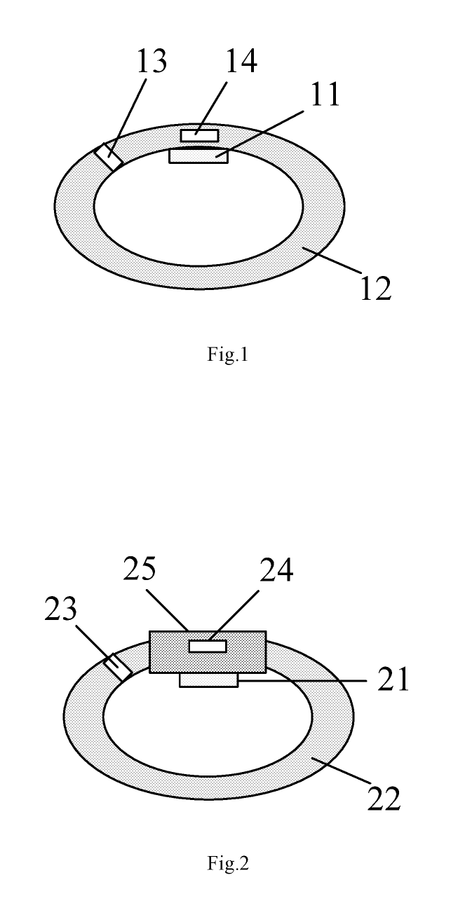

FIG. 2 schematically shows an intelligent wearable device according to another embodiment of the present invention. As shown in FIG. 2, the intelligent wearable device comprises: a photoelectric sensor 21, an inflatable ring 22, an air valve 23, a control unit 24 and a dial plate 25; wherein the air valve 23 is within the inflatable ring 22, and when the air valve 23 is in an open state, the inflatable ring 22 can be inflated and deflated alternately, and when the air valve 13 is in a closed state, inflation or deflation of the inflatable ring 22 is stopped; the photoelectric sensor 21 is located at an inner side of the inflatable ring 22 for measuring physical signs; the dial plate 25 is connected to the photoelectric sensor 21 and the inflatable ring 22 respectively for displaying various data measured by the photoelectric sensor 21; the control unit 14 is located in the dial plate 25 and is connected to the photoelectric sensor 21 and the air valve 23 respectively for controlling the state of open and close of the air valve 23 according to the physical sign parameters measured by the photoelectric sensor 21.

The intelligent wearable device as shown in FIG. 2 differs from FIG. 1 by adding a dial plate 25 for displaying various physical sign parameters and data measured by the photoelectric sensor 11 to be viewed by the user.

Preferably, as shown in FIG. 2, the dial plate 25 can be specifically provided at an outer side of the inflatable ring 22 to be easily seen by the user. Further, the dial plate 25 may comprise a display screen for displaying measured data to be viewed easily by the user.

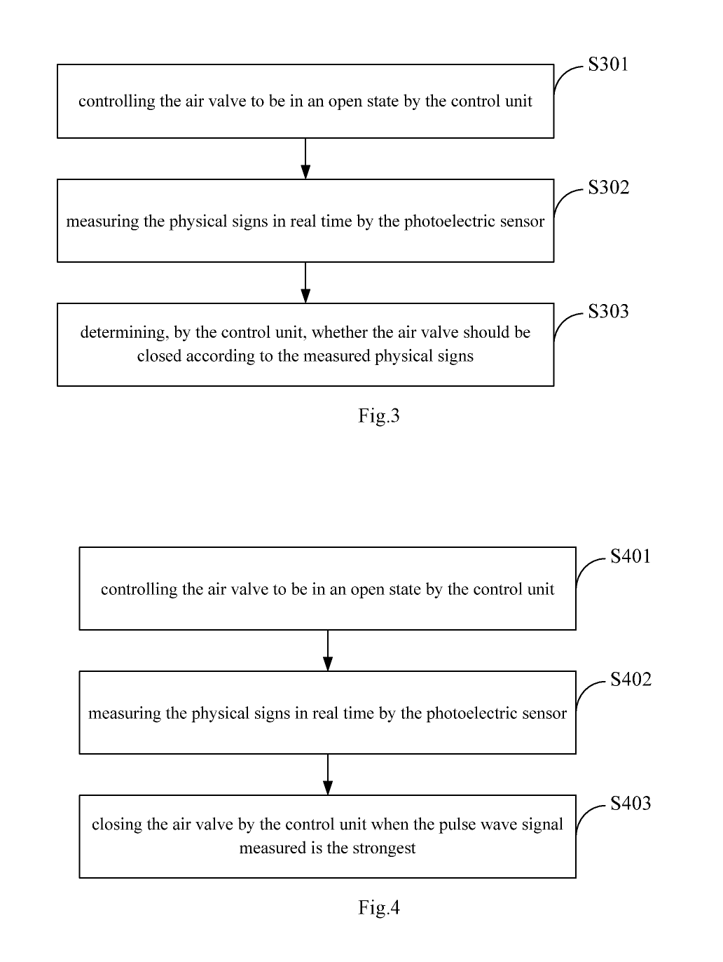

FIG. 3 is a flow chart of a method for physical sign measurement according to one embodiment of the present invention, which uses using the intelligent wearable device according to the present invention. As shown in FIG. 3, the method for physical sign measurement comprises the following steps:

S301: controlling the air valve to be in an open state by the control unit so as to inflate and deflate the inflatable ring alternately;

S302: measuring the physical signs in real time by the photoelectric sensor; and

S303: determining, by the control unit, whether the air valve should be closed according to the physical sign parameters measured by the photoelectric sensor.

In the above method for physical sign measurement, it can be set in step S301 that inflation and deflation are carried circularly and automatically and they alternate repeatedly; and in step S303, when the air valve is closed, the air valve will stop inflating or deflating to keep the inflating volume of the inflatable ring unchanged. The automatic circulating as mentioned above may be inflating and deflating in a range around the optimal inflating volume corresponding to the strongest pulse so as to find the optimal inflating volume. For example, it may be pre-set that after three times of circulating, the circulating stops when the optimal inflating volume is reached.

Preferably, in the method for physical sign measurement according to the present embodiment, the step S303 of determining, by the control unit, whether the air valve should be closed according to the physical sign parameters measured by the photoelectric sensor may include: closing the air valve by the control unit when the pulse wave signal measured by the photoelectric sensor is the strongest, wherein, the pulse wave signal being the strongest may specifically mean that the amplitude of the pulse wave is the largest. This preferred case is now described in detail with reference to FIG. 4.

FIG. 4 is a flow chart of a method for physical sign measurement provided by another embodiment of the present invention, which uses the intelligent wearable device according to the present invention. Referring to FIG. 4, the method for physical sign measurement according to the present embodiment comprises the following steps:

S401: controlling the air valve to be in an open state by the control unit so as to inflate and deflate the inflatable ring alternately;

S402: measuring the physical signs in real time by the photoelectric sensor; and

S403: closing the air valve by the control unit when the pulse wave signal measured by the photoelectric sensor is the strongest.

Preferably, in the method for physical sign measurement as shown in FIG. 4, the step S403 of closing the air valve by the control unit when the pulse wave signal measured by the photoelectric sensor is the strongest may include: during inflation of the inflatable ring, determining, by the control unit, an inflating volume (i.e. an optimal inflating volume) for the inflatable ring when the pulse wave signal is the strongest, and during deflation of the inflatable ring, closing, by the control unit, the air valve when the inflatable ring has reached the inflating volume. In the above step, the control unit may determine, in any inflation process of the inflatable ring, the optimal inflating volume of the inflatable ring when the pulse wave signal is the strongest. For example, it may determine the optimal inflating volume in the first inflation process.

Likewise, step S403 as shown in FIG. 4 may also include: during deflation of the inflatable ring, determining, by the control unit, an inflating volume (i.e. an optimal inflating volume) for the inflatable ring when the pulse wave signal is the strongest, and during inflation of the inflatable ring, closing, by the control unit, the air valve when the inflatable ring has reached the inflating volume. Wherein, the control unit may determine, in any deflation process of the inflatable ring, the optimal inflating volume of the inflatable ring when the pulse wave signal is the strongest. For example, it may determine the optimal inflating volume in the first deflation process.

Those skilled in the art should understand that all or part of the steps in the above embodiments can be carried out by hardware or by a program that instructs relevant hardware; the program may be stored in a computer-readable storage medium, and the storage medium may be a read-only memory, a magnetic disc or an optical disc, etc.

The above described are merely preferred embodiments of the present invention, but they do not intend to limit the present invention. Any modification, equivalent substitution or improvement made under the spirit and principle of the present invention shall fall within the protection scope of the present invention.

1-10. (canceled)

11. An intelligent wearable device, wherein the device comprises:
a photoelectric sensor, an inflatable ring, an air valve and a control unit;
wherein the air valve is within the inflatable ring, and when the air valve is in an open state, the inflatable ring can be inflated and deflated alternately, and when the air valve is in a closed state, inflation or deflation of the inflatable ring is stopped;
wherein the photoelectric sensor is located at an inner side of the inflatable ring for measuring physical signs; and
wherein the control unit is connected to the photoelectric sensor and the air valve respectively for controlling the state of open and close of the air valve according to the physical sign parameters measured by the photoelectric sensor.

12. The device according to claim 1, wherein the control unit is further used for, in the case where the air valve is in an open state, closing the air valve when the pulse wave signal measured by the photoelectric sensor is the strongest.

13. The device according to claim 12, wherein the control unit is further used for,
during inflation of the inflatable ring, determining an optimal inflating volume for the inflatable ring when the pulse wave signal is the strongest, and during deflation of the inflatable ring, closing the air valve when the inflatable ring has reached the optimal inflating volume.

14. The device according to claim 12, wherein,
during deflation of the inflatable ring, the control unit determines an inflating volume for the inflatable ring when the pulse wave signal is the strongest, and during inflation of the inflatable ring, the control unit closes the air valve when the inflatable ring has reached the inflating volume.

15. The device according to claim 12, wherein, when the pulse wave signal is strongest, the amplitude of the pulse wave is the largest.

16. The device according to claim 13, wherein when the pulse wave signal is the strongest, the amplitude of the pulse wave is the largest.

17. The device according to claim 14, wherein when the pulse wave signal is the strongest, the amplitude of the pulse wave is the largest.

18. The device according to claim 11, wherein the device further comprises a dial plate connected to the photoelectric sensor and the inflatable ring respectively for displaying various physical sign parameters and data measured by the photoelectric sensor, and wherein the control unit is in the dial plate.

19. A method for physical sign measurement using the intelligent wearable device according to claim 11, wherein the method comprises:
controlling the air valve to be in an open state by the control unit;
measuring the physical signs in real time by the photoelectric sensor; and
determining, by the control unit, whether the air valve should be closed according to the physical sign parameters measured by the photoelectric sensor.

20. A method for physical sign measurement using the intelligent wearable device according to claim 12, wherein the method comprises:
controlling the air valve to be in an open state by the control unit;
measuring the physical signs in real time by the photoelectric sensor; and
determining, by the control unit, whether the air valve should be closed according to the physical sign parameters measured by the photoelectric sensor.

21. A method for physical sign measurement using the intelligent wearable device according to claim 13, wherein the method comprises:
controlling the air valve to be in an open state by the control unit;
measuring the physical signs in real time by the photoelectric sensor; and
determining, by the control unit, whether the air valve should be closed according to the physical sign parameters measured by the photoelectric sensor.

22. A method for physical sign measurement using the intelligent wearable device according to claim 14, wherein the method comprises:
controlling the air valve to be in an open state by the control unit;
measuring the physical signs in real time by the photoelectric sensor; and
determining, by the control unit, whether the air valve should be closed according to the physical sign parameters measured by the photoelectric sensor.

23. A method for physical sign measurement using the intelligent wearable device according to claim 15, wherein the method comprises:
controlling the air valve to be in an open state by the control unit;
measuring the physical signs in real time by the photoelectric sensor; and
determining, by the control unit, whether the air valve should be closed according to the physical sign parameters measured by the photoelectric sensor.

24. A method for physical sign measurement using the intelligent wearable device according to claim 18, wherein the method comprises:
controlling the air valve to be in an open state by the control unit;
measuring the physical signs in real time by the photoelectric sensor; and
determining, by the control unit, whether the air valve should be closed according to the physical sign parameters measured by the photoelectric sensor.

25. The method according to claim 19, wherein the step of determining, by the control unit, whether the air valve should be closed according to the physical sign parameters measured by the photoelectric sensor comprises:
closing the air valve by the control unit when the pulse wave signal measured by the photoelectric sensor is the strongest.

26. The method according to claim 25, wherein the step of closing the air valve by the control unit when the pulse wave signal measured by the photoelectric sensor is the strongest comprises:
during inflation of the inflatable ring, determining, by the control unit, an optimal inflating volume for the inflatable ring when the pulse wave signal is the strongest, and during deflation of the inflatable ring, closing, by the control unit, the air valve when the inflatable ring has reached the optimal inflating volume; or
during deflation of the inflatable ring, determining, by the control unit, an optimal inflating volume for the inflatable ring when the pulse wave signal is the strongest, and during inflation of the inflatable ring, closing, by the control unit, the air valve when the inflatable ring has reached the optimal inflating volume.

27. The method according to claim 25, wherein, when the pulse wave signal is strongest, the amplitude of the pulse wave is the largest.

28. The method according to claim 26, wherein, when the pulse wave signal is strongest, the amplitude of the pulse wave is the largest.
